(19) United States
(12) Patent Application Publication
(10) Pub. No.: US 2016/0199579 A1
(43) Pub. Date: Jul. 14, 2016

(54) NEEDLE FREE INJECTORS

(71) Applicants: Brooks Boyd, Berkeley, CA (US); Stephen J. Farr, Orinda, CA (US); Jeffery A. Schuster, Bolinas, CA (US); Andy Fry, Hertfordshire (GB); Andy Pocock, Hertfordshire (GB); Brennan Miles, Hertfordshire (GB); Chris Hurlstone, Essex (GB); Joe Daintrey, Hertfordshire (GB)

(72) Inventors: Brooks Boyd, Berkeley, CA (US); Stephen J. Farr, Orinda, CA (US); Jeffery A. Schuster, Bolinas, CA (US); Andy Fry, Hertfordshire (GB); Andy Pocock, Hertfordshire (GB); Brennan Miles, Hertfordshire (GB); Chris Hurlstone, Essex (GB); Joe Daintrey, Hertfordshire (GB)

(73) Assignee: ZOGENIX, INC., Emeryville, CA (US)

(21) Appl. No.: 13/823,003

(22) PCT Filed: Jan. 9, 2012
(86) PCT No.: PCT/US12/20654

§ 371 Date: Mar. 22, 2016

Related U.S. Application Data
(60) Provisional application No. 61/431,325, filed on Jan. 10, 2011.

(51) Int. Cl. A61M 5/30; A61M 5/20

(52) U.S. Cl. CPC A61M 5/30; A61M 5/2053; A61M 2005/3104

(57) ABSTRACT

Improved needle free injectors comprising of a energy sources, triggering mechanisms, impact members, and drug delivery pistons are disclosed. In one preferred embodiment, the triggering mechanism comprises a spool which seals an energy source comprised of compressed gas and a component for releasing the spool to release the pressurized gas and urge a ram forward to force a drug containing formulation through a drug delivery orifice. The device may include a cap covering the orifice and safety mechanisms to prevent accidental delivery

### FIELD OF THE INVENTION

The present invention relates to needle free injectors, techniques for improving the reliability and manufacturability of needle free injectors, and needle free injectors capable of delivering increased doses.

### BACKGROUND OF THE INVENTION

Many patients are needle-averse or suffer from needle-phobia or have fear of self-administration of a needle-based medical injection. Many patients and/or health-care providers have other difficulties including inability or lack of desire to follow complex instructions, and danger of needle stick injury and cross contamination. Ensuring treatment compliance can be problematic. In addition, it is a problem that patients may need to be trained to self administer an injection, although for some indications the number of injections they would self administer is only a few. In addition, a needle and syringe in general needs to be filled, and for some formulations, dried drug requires reconstitution, which further complicates self administration and reduces compliance. These issues often rule out the possibility of treatment in a home setting, either self treatment or by a relatively un-trained care giver such as a family member. The inability to dose at home can lead to higher costs of therapy, delay in treatment, reduced compliance, reduced comfort, and potential exposure to hospital acquired infections.

A number of biologically-active agents in viscous formulations would benefit from being delivered using the needle-free injector. This group could consist of (but not limited to) anti-inflammatory agents, antibacterial agents, antiparasitic agents, antifungal agents, antiviral agents, anti-neoplastic agents, analgesic agents, anaesthetics, vaccines, central nervous system agents, growth factors, hormones, antihistamines, osteoinductive agents, cardiovascular agents, anti-ulcer agents, bronchodilators, vasodilators, birth control agents and fertility enhancing agents, interferon alpha, growth hormone, osteoporosis drugs including PTH and PTH analogs and fragments, obesity drugs, psychiatric drugs, anti-diabetes, female infertility, AIDS, treatment of growth retardation in children, hepatitis, multiple sclerosis, migraine headaches, and allergic reactions.

### SUMMARY OF THE INVENTION

An aspect of the invention is a needle-free injector which is comprised of pressurized gas cylinder which gas cylinder is not completely enclosed in the absence of a spool and seal. A spool comprised of a storage seal maintains the glass cylinder in a pressurized state during storage. The injector includes a means for releasing the spool in a manner which releases the pressurized gas into a chamber. A ram is slidably positioned in the chamber in a manner such that the ram is urged forward by released pressure from the gas cylinder. A drug container holds a liquid drug formulation in fluid connection with a drug delivery orifice. When the ram is forced to move by released pressurized gas it causes the liquid formulation to be extruded through the drug delivery orifice in a narrow jet at sufficient speed to puncture human skin and provide for a needle-free injection of the liquid drug formulation.

An aspect of the invention is that the pressurized cylinder need not be punctured due to the presence of the spool valve.

Another aspect of the invention is that the device does not require a spacer to provide an air gap between the nozzle and the injection site on the human skin.

Another aspect of the invention is that the device includes a safety feature such that the device is not accidentally triggered when the cap is removed.

Another aspect of the invention is that the device can provide for subcutaneous injection.

Another aspect of the invention is the screw cap safety feature which when removed does not trigger the device. The cap may be screwed to the drug container to ensure a good seal is maintained. In the absence of some sort of safety device the act of unscrewing the cap if combined with pushing the cap towards the rest of the device could trigger the device. However, the device includes a second set of threads on the cap that engage the cap such that when the cap is unscrewed it is also driven away from the device. This arrangement of the second set of threads on the cap can make it possible to eliminate the need for a safety mechanism such as a block actuated by a lever and makes the device simpler to use.

In one aspect of the invention the spool further comprises an additional seal that seals against loss of the pressurized gas after the gas has been released into the chamber. Further, the spool may be configured such that the pressurized gas holds the spool in a first position by a movable body which blocks motion of the spool prior to releasing the spool.

An aspect of the invention includes a means for releasing the spool so that the spool moves a movable body thereby exposing an end of a spool to a recess into which recess the spool is moved by force applied by the pressurized gas. The moveable body may be moved by the act of pressing the drug delivery orifice of the device against a surface such as human skin.

An aspect of the invention includes an injector configured such that upon releasing the spool a sub-cutaneous injection occurs forcing the liquid drug formulation out of the drug orifice and through the human skin at the injection site.

In one aspect of the invention is provided a needle-free injector which is comprised of a drug capsule containing a liquid drug formulation. The device includes an orifice in the container and the orifice leads to the liquid drug formulation in a fluid connecting manner. A first gas reservoir containing a first pressurized gas at a first pressure is used and the first pressurized gas is in contact with an urges a drug dispensing member forward. Movement of the drug dispensing member is prevented by a trigger mechanism.

A second gas reservoir containing a second pressurized gas at a second pressure is also present wherein the dispensing member is not urged forward by the second pressurized gas until after it is released by the trigger mechanism.

The invention may be carried out utilizing a pre-filled, self contained, single use, hand-held needle free injector

In a particularly preferred embodiment, the invention is carried out using a needle free injector that is powered by a self contained compressed gas charge, elements of which are described in U.S. Pat. No. 5,891,086 (incorporated by reference in its entirety). This embodiment includes a device for delivering formulations by needle-free injection, for example sub-cutaneously (SC), intra-dermally (ID) or intra-muscularly (IM). An energizer is used in conjunction with a drug cartridge to form a needle-free injector. The cartridge is pre-filled with a liquid to be injected in a subject, the cartridge having at least one liquid outlet and a free piston inward of the liquid outlet in contact with the liquid.

The energizer comprises:
(a) a housing having a forward portion adapted to be connected with the cartridge;
(b) impact member mounted within said housing inward of the forward portion so as to be movable from a first position toward the forward portion to strike the free piston when a cartridge is connected and to continue to move the free piston toward the liquid outlet whereby a dose of the liquid is expelled through the liquid outlet in the cartridge;
(c) an element within said housing which engages said impact member to prevent movement of the impact member during storage and handling, wherein upon actuation the element allows movement of the impact member.
(d) a cap that covers the injection orifice or orifices, keeping the orifice clean and ensuring the sterility of the drug formulation;
(e) a safety mechanism that ensures that the device does not actuate prematurely; and
(f) an actuator for said safety mechanism, said actuator being accessible to the user only after the orifice cap is removed, to ensure that the act of removing the orifice cap does not accidentally cause the device to fire, or alternatively:
(f) wherein the safety mechanism comprises a feature by which the act of removing the cap is actively prevented from accidentally triggering the device.

The current invention describes various formulations that can be delivered using a needle-free injector including the injector of U.S. Pat. No. 5,891,086. These formulations active ingredients, and may include various polymers, carriers, etc.

An aspect of the invention is a desirable delivery time, especially for high viscosity formulations. Desirable delivery times may include any delivery times wherein the formulation is successfully delivered. Preferred delivery times include those less than the reaction time of a human, for example less than ˜600 ms, more preferably less than 400 ms, most preferably less than 100 ms per each 0.5 mL of formulation delivered.

Another aspect of the invention is acceptable pain associated with injection

Another aspect of the invention relates to alleviation of fear of needles associated with injection of formulations.

Another aspect of the invention relates to the elimination of the danger of needle stick injury and cross-contamination associated with injection of formulations.

Another aspect of the invention relates to the simplification of preparation associated with injection of formulations, by supplying a pre-filled, single use disposable injector.

Another aspect of the invention relates to the drug release profile associated with injection of high viscosity depot formulation.

Another aspect of the invention is to improve the reliability of needle free injectors.

Another aspect of the invention is to minimize the strains and concomitant deformation and loss of reliability seen in energizer elements exposed during storage to the high forces required for successful needle free injection.

Another aspect of the invention is to minimize the amount of glass forming required to create the drug container of a needle free injector, to minimize the defects in the glass and concomitant glass breakage associated therewith upon pressurization of the drug formulation.

Another aspect of the invention is to eliminate the manufacturing difficulties associated with forming small injection orifices in glass

Another aspect of the invention is to eliminate the possibility of breakage that can occur when the formulation is rapidly pressurized for delivery when a gas bubble is in proximity to an injection orifice formed in glass.

Another aspect of the invention is to improve the manufacturability of needle free injectors.

Another aspect of the invention is to enable delivery of higher doses using needle free injection.

Another aspect of the invention is to enable the use of lower gas pressures for the power source of needle free injectors.

Another aspect of the invention is to provide a needle free injector that is very simple to use, with a simple instruction set and minimal number of steps for preparation and delivery, and requiring only basic manual dexterity and hand strength.

Another aspect of the invention is to provide a needle free injector with safety features that eliminate the possibility of accidental actuation during storage or preparation for delivery.

Another aspect of the invention is to provide a needle free injector with a cover for the injection orifice or orifices that maintains each orifice in a clean and sterile state, and maintains the sterility of the drug formulation, until the device is prepared for delivery.

Another aspect of the invention is to provide a means to ensure that the steps for preparing the injector for delivery must be carried out by the user in the correct order, for example that the orifice cap must be removed prior to, or at the same time as, removal of the safety, to ensure, for example, that the act of removing the cap does not trigger the device.

Another aspect of the invention is the elimination of the need for priming the needle free injector by causing the piercing of a hermetically sealed gas cartridge.

Another aspect of the invention is the elimination of the high variation of pressure with temperature of a power source which is comprised of a pierceable, hermetically sealed CO₂ cartridge.

Another aspect of the invention is the elimination of the additional parts and complexity associated with a gas cartridge that must be impaled on a piercing member to release the gas and deliver the medicament from a needle free injector.

These and other objects, advantages, and features of the invention will become apparent to those persons skilled in the art upon reading the details of the formulations and methodology as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to scale. On the contrary, the dimensions of the various features are arbitrarily expanded or reduced for clarity. Included in the drawings are the following figures:

FIG. 1 is a depiction of a preferred embodiment of the invention, with spool valve, shear pin, and separate nozzle.

FIG. 2 is a more detailed look at the gas cylinder, spool valve, and ram head of the embodiment of the invention shown in FIG. 1.

FIG. 3 is a more detailed look at the ram guide, piston, drug capsule, and orifice cap of the embodiment of the invention shown in FIG. 1.

FIG. 4 shows another embodiment of the invention, with a two pressure gas cylinder and ball bearing trigger.

FIG. 5 shows another embodiment of the invention, with a frangible gas cylinder seal and combined capsule and ram cylinder.

FIG. 6. shows another embodiment of the invention, with a Belleville washer stack power source, and sheet metal strut trigger.

FIG. 7 shows another embodiment of the invention, with a central mechanical spring and rear trigger assembly.

FIG. 8 shows another embodiment of the invention whereby the gas pressure acts directly on the piston, vs. via the ram as shown in other embodiments.

FIG. 9 shows another embodiment of the invention, with a rotating ram.

FIG. 10 shows another embodiment of the invention, with a hollow ram.

FIG. 11 shows a bench top prototype designed to study the dynamics of the embodiment shown in FIG. 1.

FIG. 12a shows two sample formulation pressure profiles generated with the prototype of FIG. 11 utilizing a 1 mL steel drug capsule.

FIG. 12b shows a sample formulation pressure profile generated with the type of device described in '086.

FIG. 13 shows a sample formulation pressure profile generated with the prototype of FIG. 11 utilizing a 0.5 mL glass capsule similar to that used in the device that generated the formulation pressure profile presented in FIG. 12b.

FIG. 14 shows a bench top prototype designed to study the dynamics of the two pressure gas cylinder embodiment of the invention such as that shown in FIG. 4.

FIG. 15 shows a sample formulation pressure profile generated with the prototype of FIG. 14.

### DETAILED DESCRIPTION OF THE INVENTION

Before the present formulations and methods are described, it is to be understood that this invention is not limited to particular formulations and methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. Thus, for example, reference to “a formulation” includes a plurality of such formulations and reference to “the method” includes reference to one or more methods and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

Active Pharmaceutical Ingredient, API, active drug substance, medicament, or the like: A component of a pharmaceutical formulation that is pharmaceutically active and is delivered for a desired effect.

Actuator: A mechanical device for moving or controlling a mechanism or system. An example of an actuator is a lever that a patient uses to ready an autoinjector for delivery.

Aggregation: formation of linked molecules held together by van der Waals forces or chemical bonds.

AUC: Area under the curve, or the integral, of the plasma concentration of delivered drug over time

Belleville Washers, Belleville Washer Stack, Belleville Spring, or the like: a power source for needle free injection made from a plurality of frustro-conically shaped washers which have a spring characteristic and store power when compressed. The name comes from the inventor, Jullian F. Belleville.

Biodegradable: capable of chemically breaking down or degrading within the body to form nontoxic components. The rate of degradation of a depot can be the same or different from the rate of drug release.

Biologic: A medicinal product created by biological processes (as opposed to chemically). Examples include vaccines, blood and blood components, allergenics, somatic cells, gene therapy, tissues, stem cells, immune globulins, and recombinant therapeutic proteins. Biologics may be isolated from natural sources such as humans, animals, plants, or microorganisms or may be produced by biotechnology methods.

Carbon Dioxide, or CO₂: a colorless gas that is odorless at pressures usually found in the atmosphere. CO₂ is often used as the power source for needle free injectors. CO₂ has the advantages that it is commercially available in pressurized hermetically sealed containers. The CO₂ in these containers is liquefied, and thus maintains a relatively constant pressure as the container is depleted (approximately 853 PSI at 70° F.). A disadvantage of CO₂ is the relatively large variation of pressure with temperature.

Carrier: a non-active portion of a formulation which may be a liquid and which may act as a solvent for the formulation, or wherein the formulation is suspended. Useful carriers do not adversely interact with the active pharmaceutical ingredient and have properties which allow for delivery by injection, specifically needle free injection. Preferred carriers for injection include water, saline, and mixtures thereof. Other carriers can be used provided that they can be formulated to create a suitable formulation and do not adversely affect the active pharmaceutical ingredient or human tissue.

Centipoise and centistokes: different measurements of viscosity, which are not just different units. Centipoise is a dynamic measurement of viscosity whereas centistoke is a kinematic measurement of viscosity. The conversion from centistoke and centipoise to s.i. units is given below:
1 cS=0.0001 m²/s 1 cP=0.001 Ns/m²

Coefficient of Thermal Expansion, Thermal Expansion Coefficient, and the like: The fractional change in size of a material (ΔL/L), per degree C.

Coefficient of Friction: a constant of proportionality relating the normal force between two materials and the frictional force between those materials. Generally friction is considered to be independent of other factors, such as the area of contact. The coefficient of static friction characterizes the frictional force between to materials when at rest. This force is generally what is required to start relative movement. The coefficient of dynamic friction characterizes the frictional force between to materials that are moving relative to one another. In general, the coefficient of static friction is higher than the coefficient of dynamic friction.

Container Closure, Container Closure System, Drug Container, Capsule, and the like: A drug container that is designed to maintain sterility and eliminate the possibility of contamination of the drug formulation. For container closure systems that contain aqueous formulations, the container closure system must also have sufficiently low water vapor transmission rate such that the concentration of the formulation does not change appreciably over the product shelf life. Preferred materials have sufficiently low leachable materials such that they do not comtaminate the formulation during storage. Preferred materials for container closures include glass, more preferably boro-silicate glass, or fluorinated materials such as polytetrafluoroethylene (PTFE).

Container Closure Integrity: The ability of a container closure system to maintain sterility, eliminate the possibility of contamination, and minimize loss of carrier during storage.

CPV trial: a 400 subject trial used to validate the predictive power of the IVIVC of the present invention.

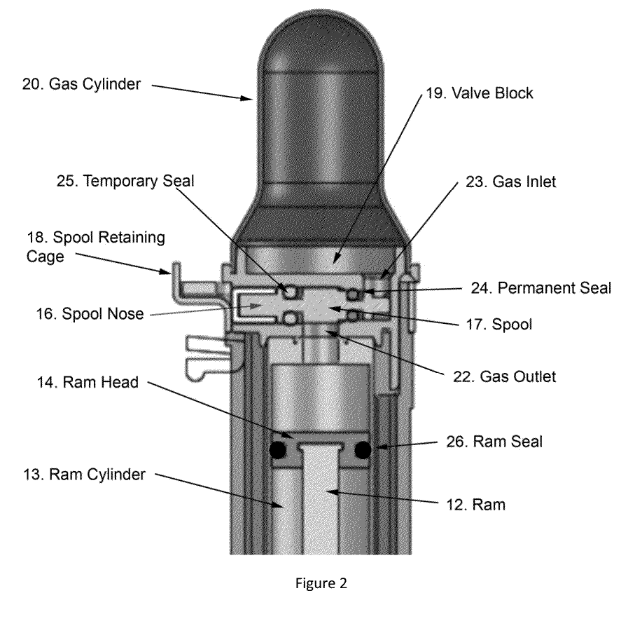

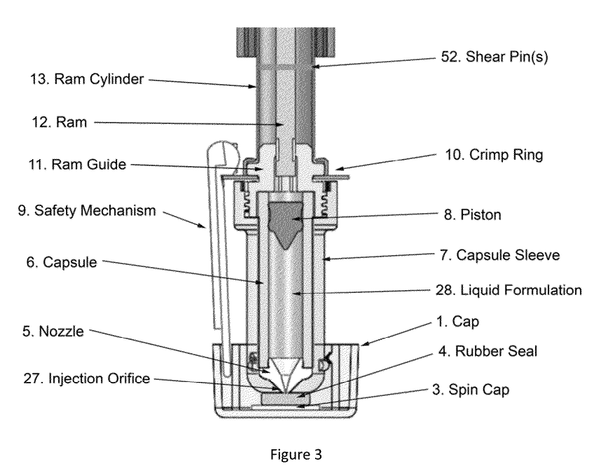

Delivery Phase: A constant or slowly varying formulation pressure during which the bulk of a formulation dose is delivered from a needle-free injector (see FIG. 2). In a preferred embodiment of the current invention, the desired injection is a subcutaneous injection. This in general requires a previous, higher pressure phase (see “puncture phase”) wherein the hole through which the injectate is delivered is formed.

Depot Injection, Depot, and the like: an injection, usually subcutaneous, intravenous, or intramuscular, of a pharmacological agent which releases its active compound in a consistent way over a long period of time. Depot injections may be available as certain forms of a drug, such as decanoate salts or esters. Examples of depot injections include Depo Provera and haloperidol decanoate. Depots can be, but are not always, localized in one spot in the body.

DosePro, Intraject, '086 system, and the like: a single use, prefilled, disposable, needle free injector currently manufactured by Zogenix corporation. A cartridge is pre-filled with a liquid to be injected in a subject, and having a liquid outlet and a free piston in contact with the liquid The injector comprises an energizer comprising an impact member urged by a compressed gas spring and temporarily restrained until the device is actuated, the impact member being movable in a first direction under the force of the spring to first strike the free piston and then to continue to move the piston in the first direction to expel a dose of liquid through the liquid outlet, the spring providing a built-in energy store and being adapted to move from a higher energy state to a lower energy state, but not vice versa. The energizer may comprise a trigger means to actuate the device, and thus initiate the injection, only when the device is pressed against the skin. Elements and variations of DosePro are described in U.S. Pat. No. 5,891,086 ('086), and additional description, improvements, and variants can be found in U.S. Pat. No. 6,620,135, U.S. Pat. No. 6,554,818, U.S. Pat. No. 6,415,631, U.S. Pat. No. 6,409,032, U.S. Pat. No. 6,280,410, U.S. Pat. No. 6,258,059, U.S. Pat. No. 6,251,091, U.S. Pat. No. 6,216,493, U.S. Pat. No. 6,179,583, U.S. Pat. No. 6,174,304, U.S. Pat. No. 6,149,625, U.S. Pat. No. 6,135,979, U.S. Pat. No. 5,957,886, U.S. Pat. No. 5,891,086, and U.S. Pat. No. 5,480,381, incorporated herein by reference.

Energizer: the mechanical portion of an autoinjector that provides the energy for injection, triggers the device, and ensures the proper pressure profile during delivery. The energizer may contain a safety mechanism that must be set prior to delivery. Note that in some prior art this portion is referred to as the actuator. However here we refer to it as the energizer to avoid confusion with, for example, the safety mechanism actuator.

Excipient: Any substance, including a carrier, added to an active drug substance to permit the mixture to achieve the appropriate physical characteristics necessary for effective delivery of the active drug.

Filter Paper Weight, or FPW: a measure of the amount of injectate left on the skin after a needle free injection event. To measure FPW, the non-injected material is absorbed onto filter paper, the sample is weighed, and the tare weight subtracted. If blood is seen in the sample, this is noted, and in general the results are not used as the blood will cause an overestimate of the FPW. The FPW can be used to correct the VAS, see definition of VAS and example 1.

Formulation, Injectate, and the like: Any liquid, solid, or other state of matter that can be injected. Preferred formulations are liquid formulations, including but not limited to solutions, suspensions including nano-suspensions, emulsions, polymers and gels. Formulations include but are not limited to those containing excipients that are suitable for injection, and contain one or more active pharmaceutical ingredients.

Frustro-conical: Having the shape of a cone whose tip has been truncated by a plane parallel to its base. See Belleville Washers.

Hermetically Sealed Container and the like: a container for pressurized gas used as the power source for needle free injection that is impervious to leakage of the contained gas. Commonly, hermetically sealed containers are formed from deep drawn zinc plated steel and contain pressurized gasses such as nitrogen, or liquefied gasses such as carbon dioxide or nitrous oxide. They are often used in the food service industry for such preparations as soda water or whipped cream, but also find medical applications in areas such as aerosol inhalation (c.f. U.S. Pat. No. 6,981,660) or needle free injection (c.f. us 3.10. U.S. Pat. No. 6,607,510). Usually these containers have a feature that is designed to be pierced to allow the pressurized contents to be accessed.

Immunogenicity: The ability of a substance (an antigen) to provoke an immune response. Aggregated biologic drugs can be immunogenic even when the unaggregated molecule is not immunogenic.

Impact gap, and the like: The width of a gap between an impact member (see ram) and a piston used to create a pressure spike in the formulation. During a needle free delivery event, the impact member is urged across the gap, for example by compressed gas or another energy source, wherein it integrates the work done by the energy source as it travels across the gap, and delivers this energy to the formulation upon impact, creating an early pressure spike. See also “Puncture Phase”.

In vivo (from the Latin for “within the living”): Experimentation using a whole, living organism as opposed to a partial or dead organism, or an in vitro experiment. In vivo research includes animal testing and human clinical trials. In vivo testing is often preferred over in vitro testing because the results may be more predictive of clinical results

In vitro (from the Latin for “within the glass”): A procedure not in a living organism (see in vivo) but in a controlled environment, such as in a test tube or other laboratory experimental apparatus. In vitro testing is often preferred over in vivo testing due to reduced cost and reduced danger to human and/or animal subjects.

In vivo/in vitro correlation, IVIVC, and the like: a model, preferably a mathematical model, that predicts in vivo performance based on in vitro measurements, design parameters, and the like. A predictive IVIVC allows the predictive value of in vivo measurements without the need for expensive and potentially dangerous human or animal clinical trials. An IVIVC is preferably based on a meta-analysis of several clinical, preferably human, trials utilizing different configurations of a drug, drug delivery technology, or other medical device technology. For the sake of this discussion, and IVIVC can be taken to mean a model that predicts in vivo injection performance of a needle free injector based on injector design parameters and bench measurements of performance.

Jet Test, Jet Tester, Jet Test Method, and the like: a laboratory apparatus that measures the force on a transducer when impinged upon by the liquid jet during a simulated drug delivery event. Using these data the formulation pressure over time can be calculated. The Jet Test is often conducting simultaneously with the Strain Gauge test.

Needle free Injector, Needle-less injector, Jet Injector, and the like: a drug delivery system which delivers a subcutaneous, intramuscular, or intradermal injection without the use of a hypodermic needle. Injection is achieved by creating at least one high velocity liquid jet with sufficient velocity to penetrate the skin, stratum subcutaneum, or muscle to the desired depth. Needle free injection systems include, but are not limited to, the DosePro® system manufactured by Zogenix Corporation, the Bioject® 2000, Iject or Vitaject devices manufactured by Bioject Medical Technologies, Incorporated, the Mediject VISION and Mediject VALEO devices manufactured by Antares, the PenJet device manufactured by Visionary Medical, the CrossJect device manufactured by Crossject, the MiniJect device manufactured by Biovalve, the Implaject device manufactured by Caretek Medical, the PowderJect device manufactured by AlgoRx, the J-tip device manufactured by National Medical Products, the AdvantaJet manufactured by Activa Systems, the Injex 30 device manufactured by Injex-Equidyne, and the Mhi-500 device manufactured by Medical House Products.

Piston: a component of a needle free injector that under force from an energy source drives liquid formulation out of an orifice to achieve a needle free injection. In a preferred embodiment, the needle free injector is prefilled with formulation, and the piston then becomes a drug contact surface of the container-closure system. In a particularly preferred embodiment, the piston has the additional function of transmitting energy from an impact member to the formulation to create a pressure spike, see “Puncture Phase”. Preferably, the piston comprises PTFE.

Polytetrafluoroethylene, PTFE, Teflon, and the like: a synthetic fluoropolymer of tetrafluoroethylene. PTFE is most well known by the DuPont brand name Teflon. PTFE is a high molecular weight fluorocarbon solid, consisting wholly of carbon and fluorine. PTFE has one of the lowest coefficients of friction against any solid. PTFE has also been shown to be an acceptable drug contact surface for many drug formulations.

Prophylaxis: The administration of a drug used to prevent the occurrence or development of an adverse condition or medical disorder.

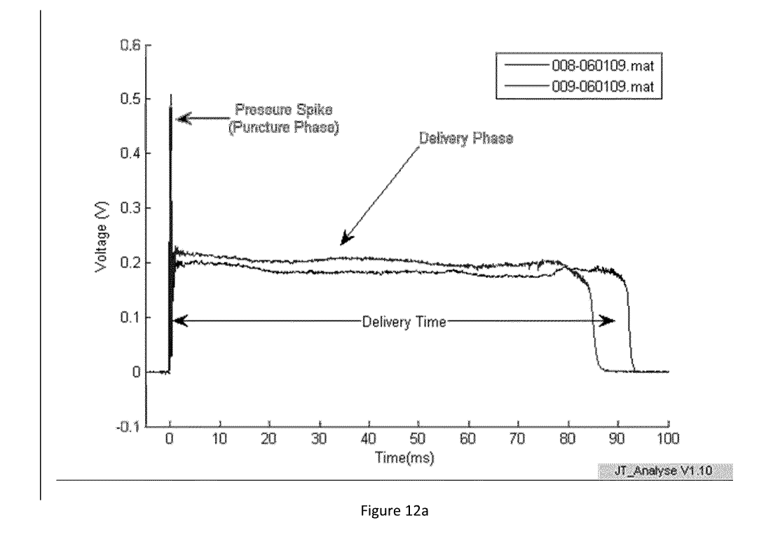

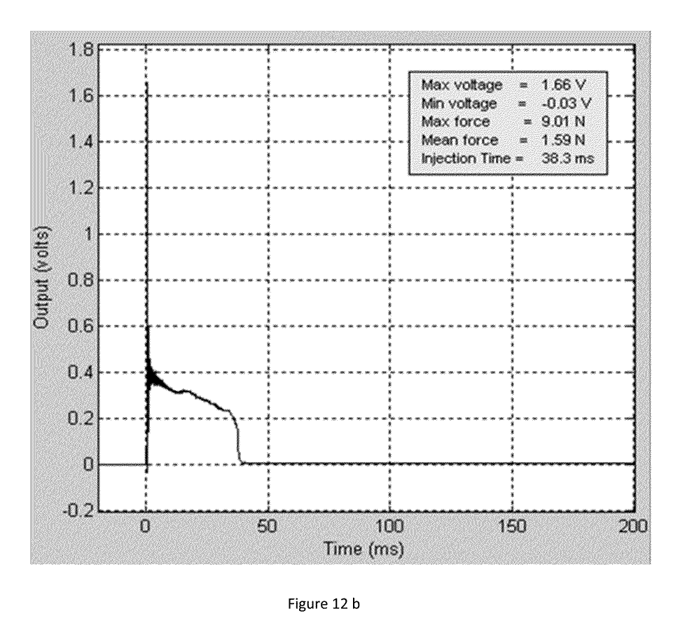

Puncture Phase, Initial Pressure Spike, and the like: An initial spike in pressure in the formulation in a needle-free injector that creates a jet with sufficient energy to drill to the desired depth into or through the skin (see FIGS. 12, 13, and 15). In a preferred embodiment of the invention, the injection is a subcutaneous injection. In order to achieve an efficient, reproducible subcutaneous injection, it is important that the jet be sufficiently energetic to drill down to the subcutaneum. However, it is then important that the bulk of the formulation be delivered at a lower pressure, in order that the formation of the hole is stopped prior to the injection becoming a painful intra-muscular injection.

Ram, impact member, and the like: a component that when exposed to a pressure is urged forward across an air space (see “impact gap”) before striking a drug delivery piston. The work done by the expanding gas as the ram traverses the impact gap is essentially all delivered to the formulation when the ram strikes the piston, creating a pressure spike (see “puncture phase”) that creates a hole in the skin to the desired depth, for example the subcutaneum. The pressurized gas then drives the ram and piston forward, delivering the formulation through the hole and into the desired tissue.

Resilient: returning to the original form or position after being bent, compressed, or stretched

Specific gravity: The ratio of a compound's density to that of water.

Spool Valve: a valve wherein the pressure of the needle-free injector pressurized gas power source urges a gas blocking component forward, but motion of the gas blocking component is inhibited by an additional device element. When the additional device component is removed, preferably due to relative movement of the additional device component when the needle-free injector is pressed against the skin of a patient, the gas blocking component is allowed to move forward, exposing a gas exit port that allows the pressurized gas to flow to a drug delivery mechanism, causing drug delivery. In one embodiment, the “balanced spool valve”, the proximal and distal ends of the gas blocking component are exposed to the power source pressure, and expose surfaces of different areas to the pressurized gas, allowing the actuation force to be tuned, and potentially optimizing and/or minimizing the frictional force on the additional device component that blocks movement of the gas blocking component.

Spring: a mechanism capable of storing energy for use in propelling the medicament in the syringe into and through the patient's skin and into body, wherein the force provided by the energy store is proportional to a displacement. This mechanism may be mechanical, e.g. compressible metal component such as a coil spring or Belleville washer stack. Preferably, the mechanism is a compressed gas spring in which the energy is stored, and when released the gas expands.

Stiff: having a high elastic modulus or low compressibility. In this case, a material that is able to transmit impact energy effectively through it medium.

Strain Gauge Test, Strain Gauge Method, and the like: A method of measuring the formulation pressure during an in vitro delivery event, wherein a strain gauge is attached to the formulation container, calibrated for formulation pressure, and then used to measure the pressure profile over time of the formulation. The Strain Gauge Test is generally conducted in parallel with a Jet Test.

Subcutaneous tissue, stratum subcutaneum, hypodermis, hypoderm, or superficial fascia, and the like: A layer of tissue that lies immediately below the dermis of skin, consisting primarily of loose connective tissue and lobules of fat. The stratum subcutaneum is the target of a subcutaneous injection.

Visual Assessment Score, VAS, and the like: A semi-quantitative method of scoring needle free injections on a scale of 0-4, based on observation. Any injection scored as a 0, 1 or 2 is termed unsuccessful (see “wet injection”, below), while a 3 or 4 is a successful injection. Injection scores are defined as follows:

0=100% splash back of injectate, not even a hole in the epidermis

1=hole in the epidermis but very little, if any penetration of injectate

2=some penetration of injectate (˜5% and <90%)

3=˜90 and <95% penetration of injectate

4=˜95% penetration of injectate

Water Vapor Transmission Rate (WVTR)) is the steady state rate at which water vapor permeates through a material. Values are expressed in g/100 in²/24 hr in US standard units and g/m²/24 hr in metric units.

Wet injection: an unsuccessful needle free injection, whereby more than 10% of the injectate does not penetrate to the stratum subcutaneum. A related definition is an injection with a Visual Assessment Score (VAS) of less than 3.

### INVENTION IN GENERAL

The current invention is related to improvements to pre-filled needle free injectors to improve reliability, safety, and manufacturability.

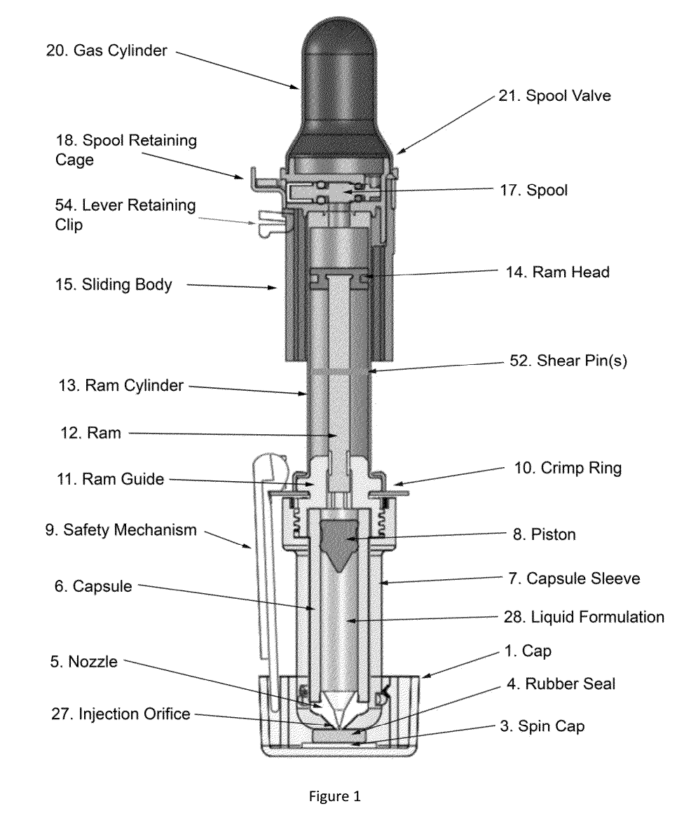

One embodiment of the invention is shown in FIG. 1. This embodiment has a number of improvements over the prior art devices.

One improvement is to gas cylinder 20. As compared to other devices, gas cylinder 20 is larger in diameter and less deeply drawn. This allows a larger volume, and thus less change in pressure as delivery progresses. At the same time, it easier to manufacture, being less deeply drawn than the gas cylinder in, for example, the device described in '086. Preferably gas cylinder 20 is deep drawn aluminum, although other fabrication techniques including but not limited to impact extrusion, die casting, or machining may be used. As shown in FIG. 1, gas cylinder 20 and valve block 19 can be separate components, but it may be desirable to combine them, using machining possibly combined with deep drawing.

The gas in gas cylinder 20 is contained during storage, and released upon triggering of the device, by spool valve 21. Unlike some prior art devices, spool valve 21 is functionally separate from the component that converts the pressure of the gas in gas cylinder 20 into the energy required to cause needle-free injection, in this embodiment ram 12. This allows the forces that spool valve 21 is subjected to during storage to be significantly less than those that ram 12 would be subjected to were it exposed to the pressurized gas during storage, due to the large differences in area exposed to the pressurized gas. This greatly minimizes the possibility of deformation and creep, and thereby reduces the possibility of premature firing or lack of firing. These issues can be exacerbated by high temperatures seen during storage or accelerated pharmaceutical stability. This aspect of the invention can remove a potential need for a device priming step that overcomes these issues.

The functioning of spool valve 21 is as follows. When the device is held by its case (not shown) and injection orifice or orifices 27 are pressed against the patient's skin at the intended injection site, sliding body 15 moves downward. This exposes spool 17 to spool retaining cage 18, which in turn allows spool 17 to move to the left as shown in FIG. 1. This exposes gas outlet 22 at the bottom of valve block 19 to the pressurized gas from gas cylinder 20 via gas inlet 23 at the top of valve block 19, allowing the gas to travel to and create a force against ram head 14.

Valve block 19 is preferably machined aluminum, but may be made by methods including but not limited to die casting, and may be combined with gas cylinder 20 and/or ram cylinder 13.

Prior art devices, such as that described in U.S. Pat. No. 6,607,510 ('510), have a hermetically sealed gas cartridge wherein the device is “primed” by impaling the cartridge on a piercing element to release the gas. In the invention disclosed in '510, an orifice cap is removed and then screwed into the opposite end of the device, forcing the hermetically sealed gas cartridge onto the piercing element. As such, any additional valve components do not require a perfect seal, such as an O-ring seal, and no such seal is disclosed in '510. However, in the embodiment described here, the spool valve is the primary seal that keeps the pressurized gas from leaking during storage, and thus requires additional sealing elements 24 and 25 in spool 17 (see FIG. 2). These sealing elements 24 and 25 may be but are not limited to o-rings or a sealing grease, but preferably are over-molded onto spool 17, or potentially one seal of each type as the requirements for permanent seal 24 are more stringent than those for temporary seal 25 which must only hold the pressure for at most a few hundred milliseconds during a delivery. Spool 17 is preferably machined brass although other materials may be used, including but not limited to other metals or polymers, and other fabrication methods may be used, including but limited to injection molding or die casting.

Spool retaining cage 18 is preferably stamped, but alternatives include but are not limited to die casting or injection molded polymers or metals.

In the device described in '086, the ram is a right circular cylinder, with the ram and perpendicular details described above. Because it is of constant and relatively small cross sectional area, the gas pressure required to create the desired formulation pressure and puncture phase pressure are quite large, creating issues around component deformation and gas leakage. To reduce the gas pressure, the pressurized gas in the embodiment of the current invention shown in FIG. 1 is introduced to ram 12 via ram head 14, which has significantly larger diameter than ram 12, see FIGS. 1 and 2. This allows the length, diameter, and mass of ram 12 to be optimized for guiding in ram guide 11 and matched to the desired travel of piston 8 in capsule 6 plus the impact gap required for the desired puncture phase pressure, while achieving the required force for the puncture phase and delivery phase at a significantly reduced gas pressure. Ram head 14 is sealed to the inside of ram cylinder 13 via ram seal 26, utilizing a sealing method including but not limited an o-ring or over-molded seal. Preferred materials for an o-ring seal are PTFE, Nitrile, or FEP coated silicone. Alternatively, ram seal 26 could be a disk or washer attached to the top ram head 14 as a one way valve or “check valve”, similar to a bicycle pump. This would allow the possibility of filling past ram head 14.

Ram 12 and ram head 14 are preferably machined from a single piece of aluminum, but alternatively may be a single cold formed piece, machined from separate parts, diecast magnesium or zinc, or an over-molded polymer head on a machined shaft. Preferably ram 12 is inserted into ram guide 11 after filling of the pressurized gas and after any leak checking and after ram cylinder 13 is attached to valve block 19, but alternatively may be assembled prior to filling if ram seal 26 is a one way valve or may be inserted into ram cylinder 13 prior to ram cylinder 13 being attached to valve block 19.

In order that ram 12 remain in place as assembled to maintain the required impact gap, ram 12 must be held either by a feature that breaks away under the force of the pressurized gas, or held in place by a frictional force that is strong enough to hold ram 12 during handling and storage but is small compared to the force of the pressurized gas bearing on ram head 14. One embodiment of this, shown in FIG. 1 is shear pin or pins 52 that break under the force of the pressurized gas on ram head 14. A related solution would be a stamped or etched crush disk mounted in ram guide 11 via a friction fit. Additional solutions include over-molded or friction fit polymer parts attached to ram guide 11.

Ram guide 11 is preferably a zinc or aluminum die casting, although other solutions include but are not limited to injection molded polymers or metals or machined steel or aluminum. Ram head 14 is guided by ram cylinder 13, and preferably ram cylinder 13 is fabricated from stock tubing, although other solutions include but are not limited to deep drawn or impact extruded, injection molded polymer, machined including machined as part of valve block 19, die cast, or extruded. Preferably ram cylinder 13 is stock tubing with swaged ends, welded to valve block 19 and attached to ram guide 11 with a crimp ring 10. Alternatives include but are not limited to welding to ram guidell and/or crimping to valve block 19.

Ram 12 is guided by ram guide 11 to strike and then drive piston 8 to deliver liquid drug formulation 28 contained within the drug container defined and closed by piston 8, capsule 6, nozzle 5, and rubber seal 4. Capsule 6 is reinforced by capsule sleeve 7, which also serves to hold nozzle 5 in contact with capsule 6 in those embodiments of the invention wherein nozzle 5 is a separate part, as shown in FIG. 1.

Capsule sleeve 7 is preferably an injection molded plastic component, but other solutions are possible, including but not limited to a steel stamping or zinc or magnesium die casting. Capsule sleeve 7 is preferably screwed onto ram guide 11, but may also be attached with a crimp ring or by other attachment methods. Capsule sleeve 7 also has additional features that allow attachment of cap 1 (see below).

The body of drug capsule 6 is preferably glass, more preferably borosilicate glass. In one embodiment, the sides of capsule 6 are simple sections of glass tubing, also known as “cane”. In this embodiment, nozzle 5 is a separate part held in place by capsule sleeve 7. This embodiment has the advantages of ease of fabrication and also has the advantage of allowing a continuous taper from the inlet of nozzle 5 to injection orifice or orifices 27, which allows for better liquid flow characteristics. Preferably, nozzle 5 is machined from a polymer, more preferably from Polytetrafluoroethylene (PTFE). Other embodiments utilize other polymers or metals and may be injection molded, die cast, machined, stamped, or utilize any other fabrication technique. Optionally, nozzle 5 may incorporate a metal support collar to minimize distortion of injection orifice 27 upon pressurization. Capsule 6 and capsule sleeve 7 are preferably assembled by inserting the optional support collar, then optional nozzle 5, and finally capsule 6 into capsule sleeve 7 with an interference fit. Injection orifice or orifices 27 are preferably machined, but may also be fabricated by a method selected from but not limited to e-beam, laser drilling, or liquid jet cutting. Optionally, the quality of an orifice created by any of the above means may be improved by an etching step, including but not limited to chemical etching, or plasma etching, or by rotating the part as an orifice is created.

In another preferred embodiment, drug capsule 6 does not have a separate nozzle 5, but instead is formed from a single piece of glass into which injection orifice or orifices 27 are fabricated. While this configuration has certain disadvantages relative to machinability, forming of the injection orifice, and breakage upon pressurization of formulation 28, it has the advantage of being developed and proven, for example in the '086 device. As with capsule 6 with separate polymer nozzle 5 embodiment above, this embodiment is assembled by inserting glass capsule 6 into capsule sleeve 7 with an interference fit. In this embodiment, injection orifice or orifices 27 are preferably laser drilled, more preferably UV laser drilled, most preferably excimer laser drilled, but may also be fabricated by a method selected from but not limited to e-beam, machining, or liquid jet cutting. Optionally, the quality and centering of orifice 27 may be improved by rotating capsule 6 while fabricating the hole. Also optionally, the quality of orifice or orifices 27 created by any of the above means may be improved by an etching step, including but not limited to chemical etching or plasma etching.

Piston 8 is preferably machined from PTFE. This has certain advantages, including the lubricious properties of PTFE, the fact that PTFE is non-reactive and thus an excellent drug contact surface, and also that PTFE is a material which is substantially non-resilient when subjected to a slowly applied force but is highly resilient when subjected to a rapidly applied force, (c.f. U.S. Pat. No. 5,891,086) allowing it to be slowly inserted into glass capsule 6 with a very tight interference fit, but allowing it to still transmit the bulk of the energy of impact of ram 12 to formulation 28 almost instantaneously.

To maintain sterility of formulation 28, limit water vapor transmission, and keep orifice or orifices 27 free of foreign debris, injection orifice or orifices 27 are preferably covered with rubber seal 4. Rubber seal 4 is attached to cap 1 through a rotating element, spin cap 3. Spin cap 3 prevents strain in and concomitant leakage from rubber seal 4 that may arise as rubber seal 4 is rotationally seated onto nozzle 5 by screwing cap 1 onto threads that are part of capsule sleeve 7.

While it is preferred that cap 1 be removed by unscrewing from threads as shown in FIG. 1, there are other methods, including but not limited to break off, click off, or not removing cap 1 but instead allowing the liquid jet to break through a bather. In one embodiment, cap 1 is attached to all or part of the secondary packaging, such as a box or polymer film overwrap, and the act of removing the device from the secondary packaging causes cap 1 to be removed, or similarly requires cap 1 to be removed.

To ensure that the device is not accidentally triggered during storage, transport, or removal of cap 1, safety mechanism 9 is included. Safety mechanism 9 blocks the movement of the case relative to the internal components, and thus prevents triggering of the device. In the embodiment shown in FIG. 1, safety mechanism 9 comprises a lever which is actuated by the user to place the device in the ready to fire state. The tip of the lever of safety mechanism 9 is captured under cap 1 (see FIG. 1) which ensures that cap 1 must be removed before the device can be placed in the ready to deliver state, eliminating the possibility of accidentally and prematurely triggering the device through the act of removing cap 1. Preferably safety mechanism 9 is fabricated of injection molded polymer and attached to the case by being captured between two clam-shell case components, although other materials, fabrication methods, and/or attachment methods are possible. In the embodiment of the invention shown in FIG. 1, safety mechanism 9 is held in place after it has been moved to the ready fire position by lever retaining clip 54. Another embodiment of safety mechanism 9 has a separate actuator lever from the component that locks the case movement. This embodiment has the advantage of being fail-safe if the separate lever component is lost.

In yet another embodiment of the device, there is no separate safety mechanism 9. Instead, cap 1 is threaded to both case 2 and capsule sleeve 7, in such a way that when it is screwed on, it bottoms out by firmly pressing the rubber seal 4 against nozzle 5 sealing the injection orifice. The threads on the case bias cap 1 and capsule sleeve 7 and therefore the internal components downward (where downward is as shown in FIG. 1) during assembly (and specifically the attachment of cap 1), storage, handling, and transport, and during removal of cap 1, ensuring the device is not accidentally triggered. This has the advantage of reduced parts count, and also renders the device easier to use as it eliminates the step of moving the lever of safety mechanism 9.

The case (not shown) is preferably a injection molded plastic clam shell assembly, preferably attached to the interior components by friction fit, although other methods of attachment, including but not limited to a snap fit, adhesives, or friction weld may be used. Preferably ram guide 11 has features that prevent the rotation of the internal components relative to the case, but alternatives include but are not limited to features on ram cylinder 13, valve block 19, features on sliding body 15, or features on capsule sleeve 7. Similarly, the case is preferably designed to interact with ram cylinder 13 to linearly guide the internal components relative to the case when injection orifice or orifices 27 are pressed against the skin, but alternatives include but are not limited to interaction with valve block 19, sliding body 15, or features on capsule sleeve 7. In addition, a reactive polymer, or more preferably a viscous or kilopoise grease is preferably included between ram cylinder 13 and the case, or alternatively between ram cylinder 13 and sliding body 15. This has numerous advantages, including
Maintaining a minimum acceptable triggering force when the device is pressed against the skin
Maintaining the correct skin stretch at actuation
Avoiding accidental triggering after setting the device in the ready to trigger state, but before delivery
Damping recoil of injection orifice 27 from the skin upon actuation.

Other methods of maintaining a minimum acceptable trigger force, maintaining skin stretch, and avoiding accidental triggering include, but are not limited to a spring or a detente between the internal components and the case. Other methods of minimizing accidental triggering include but are not limited to a retractable guard, similar to those used to prevent needle stick injury from a needle syringe.

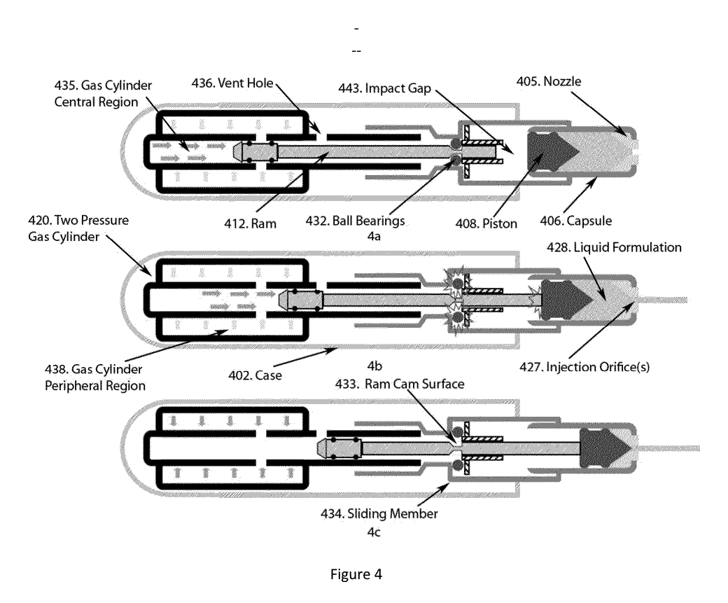

FIG. 4 shows a different embodiment of the device, with a ball bearing trigger 432, and a two pressure gas cylinder 420. Although ball bearing trigger 432 and two pressure gas cylinder 420 are shown together in FIG. 4, it is to be understood that they are independent and can be individually combined with the embodiments described above and below. The functioning of the other components, e.g. piston 408, drug capsule 406, nozzle 405, injection orifice or orifices 427, liquid formulation 428, cap (not shown), and case 402 are similar to the analogous components shown in FIG. 1 as described above.

In the ball bearing trigger embodiment shown in FIG. 4, ram 412 has a cam surface 433 machined into it that urges ball bearings 432 radially outward under the force of the pressurized gas urging ram 412 to the right as shown in FIG. 4. Sliding member 434, attached to capsule 406, captures ball bearings 432, and thus ram 412, preventing ram 412 from moving to the right as shown in FIG. 4a. After preparing the device for delivery, preferably in the way described above, nozzle 405 is pressed against the desired injection site. This causes sliding member 434 to move relative to ball bearings 432 until ball bearings 432 reach the section of sliding member 434 that no longer constrains their radial movement, as shown in FIG. 4b. Under the force exerted by cam surface 433 in ram 412, ball bearings 432 move radially, freeing ram 412. Ram 412 now flies across the impact gap 443 and strikes piston 408 as in the embodiments above, creating the pressure spike associated with the puncture phase. Ram 412 and piston 408 are then driven to the left as shown in FIG. 4c under the urging of the gas pressure, creating the delivery phase. Also as shown in FIG. 4, this embodiment has an optional vent hole 436 which enables the venting of the pressurized gas after the delivery event is completed.

FIG. 4 also shows a two pressure embodiment of the gas cylinder. In this embodiment, ram 412 is subjected to a first force during storage, triggering, and as it flies across impact gap 443, due to the pressure in gas cylinder central region 437. Subsequently during the delivery phase, ram 412 is subjected to a second, preferably lower, force which is the combined effect of the gas from central region 437 and the gas from gas cylinder peripheral region 438. This embodiment allows further independent optimization of the properties of the puncture and delivery phases. In a related embodiment (not shown), central portion 438 of gas cylinder 435 contains a mechanical rather than gas spring, such as a coil spring or Belleville washer stack. This embodiment allows for complete independence of the first and second forces if the spring crosses its zero point prior to ram 412 striking piston 408, as ram 412 will subsequently only be urged forward by the pressure of the gas in the peripheral region 438 of gas cylinder 435.

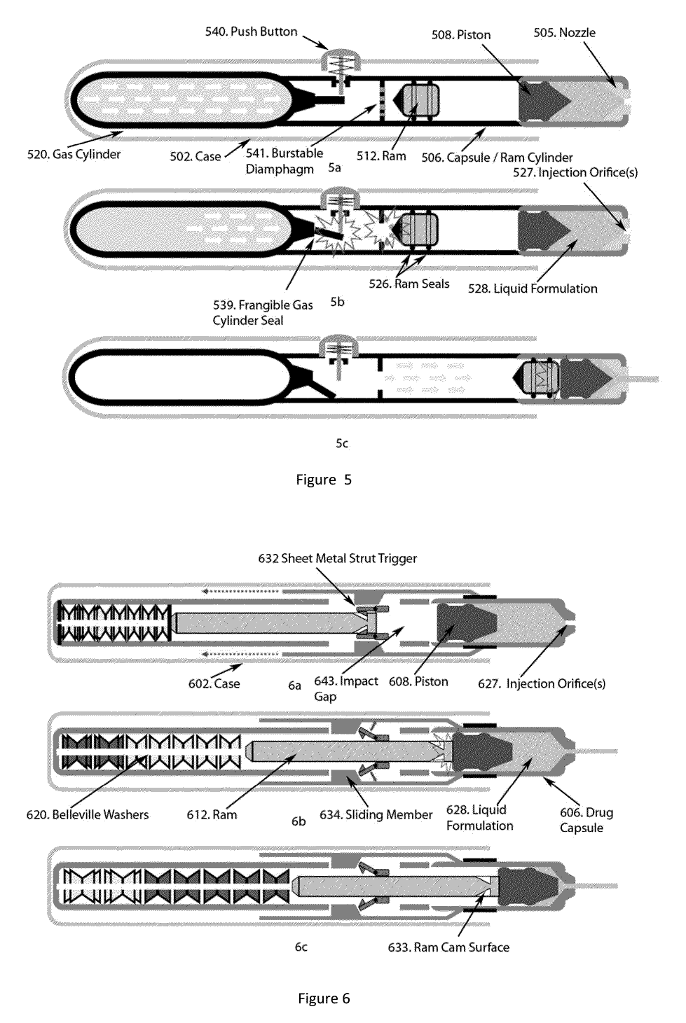

FIG. 5 shows an embodiment of the invention wherein the functions of the ram cylinder, ram guide, and glass capsule are combined in capsule/ram cylinder 506, and wherein the trigger comprises frangible gas cylinder seal 539 that is broken by push button 540. Although this trigger embodiment and ram guide/drug container embodiment are shown together in FIG. 5, it is to be understood that they are independent and can be individually combined with the embodiments described above and below. The functioning of the other components, e.g. gas cylinder 520, piston 508, nozzle 505, injection orifice or orifices 527, liquid formulation 528, cap (not shown), and case 502 are similar to the analogous components shown in FIG. 1 as described above.

In the embodiment shown in FIG. 5, the glass cane of capsule/ram cylinder 506 is extended and ram 512 is guided and sealed by a pair of ram seals 526 in contact with the glass cane. Ram 512 can be made of any material including metals or polymers. Ram seals 526 can be made in many ways, including o-rings, sealing grease, or over-molded polymer. In one embodiment, ram and seals are machined from a single piece of PTFE. In another embodiment, the ram 512 is machined brass onto which ram seals 526 are over-molded, or alternatively ram seals 526 are o-ring seals. On storage and during handling and preparation for delivery, the position of ram 512 is maintained by the air space defined by burstable diaphragm 541 to the left of ram 512 as shown in FIG. 5a, and the air space defined by ram 512 and piston 508 to the right. If ram 512 moves, an air pressure differential will arise, creating a restoring force that tends to return ram 512 to its equilibrium position. As shown in FIG. 5b, when the device is triggered the air pressure from gas cylinder 520 bursts burstable diaphragm 541, and the gas subsequently exerts a pressure on ram 512. Subsequently, ram 512, under the force of the gas pressure, is urged to the right as shown in FIG. 5c, where it strikes piston 508 creating the puncture phase, and then delivers liquid formulation 528 under the force of the pressurized gas during the delivery phase.

Also shown in FIG. 5 is the embodiment of the trigger wherein gas cylinder 530 is sealed with frangible seal 539. FIG. 5a shows the device in the ready to deliver state, with the orifice cap removed. In this embodiment, the user presses the device against the desired injection site, and then presses push button 540 to trigger. As shown in FIG. 5b, pressing push button 540 breaks frangible gas cylinder seal 539, allowing the gas to escape and triggering the device.

FIG. 6 shows an embodiment of the invention wherein the power source is compressed stack of Belleville washers 620, and with an alternate embodiment of the trigger, sheet medal strut trigger 632. Although this trigger and power source are shown together in FIG. 6, it is to be understood that they are independent and can be individually combined with the embodiments described above and below. The functioning of the other components, e.g. piston 608, injection orifice or orifices 627, liquid formulation 628, drug capsule 606, cap (not shown) and case 602 are similar to the analogous components shown in FIG. 1 as described above.

In the embodiment shown in FIG. 6, the power for injection is supplied by compressed stack of Belleville washers 620. This embodiment of the power source has certain advantages, including the fact that leakage cannot occur and the need for hermetic seals is obviated. The functioning of the system is similar to that shown in FIG. 1 and described above: FIG. 6a shows the system in the ready to deliver state, with the cap removed. When the device is triggered, Belleville washer stack 620 causes ram 612 to fly across impact gap 643 and strike piston 608, as shown in 6b, creating the pressure spike of the puncture phase. Subsequently Belleville washer stack 620 drives piston 608 via ram 612 and delivers liquid formulation 628 during the delivery phase. Use of Belleville washer stacks of differing spring constants allows for some tuning of the delivery parameters. As shown in FIG. 6b, higher rate Belleville washer stacks can be constructed by replacing individual Belleville washers with two or more nested washers. Precisely tuned spring forces can be achieved by replacing some or all of the Belleville washers with nested washers.

Also shown in FIG. 6 is an alternate embodiment of the trigger, sheet metal strut trigger 632. This embodiment is somewhat similar to the ball bearing trigger described above, but with ball bearings 432 replaced by sheet metal strut trigger 632 shown in FIG. 6. In FIG. 6a, the device is shown in the ready to trigger state. Cam surfaces 633 on ram 612 urge the sheet metal struts 632 outward, but their movement is blocked by sliding member 634 that is mechanically attached to drug capsule 606. When the device is pressed against the desired injection site, sliding member 634 moves to the left as shown in FIG. 6b, removing the constraint of sliding member 634 holding struts 632 in place, which in turn allows struts 632 to move radially outward under the force of cam surfaces 633, freeing ram 612 and triggering the device. Ram 612 now flies across the impact gap 643 and strikes piston 608 as in the embodiments above, creating the pressure spike associated with the puncture phase

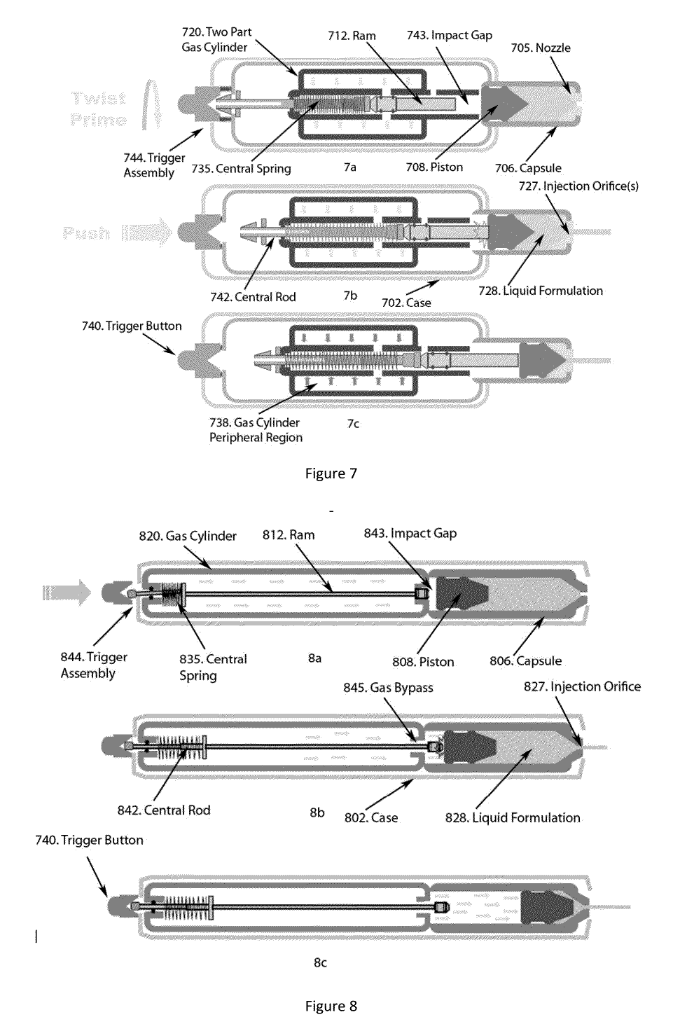

FIG. 7 shows an additional embodiment of the device. In this embodiment, somewhat related to that shown in FIG. 4 and described above, the power source is a two part gas cylinder 720 with a central mechanical spring 735 (shown) or gas spring (not shown). The functioning of the other components, e.g. ram 712, piston 708, capsule 706, nozzle 705, injection orifice or orifices 727, liquid formulation 728, cap (not shown) and case 702 are similar to those shown in FIG. 1 as described above. Here central spring 735 is wound on and captured by central rod 742 which is attached to trigger assembly 744 at the left of the device, with actuating trigger button 740, as shown in FIG. 7a. To prevent accidental triggering, trigger button 740 is rotated immediately prior to delivery to place the device in the ready to deliver state. Nozzle 705 is then pressed against the desired delivery site, and trigger button 740 is pressed to release central rod 742, which then drives ram 712 to the right across impact gap 743, as shown in FIG. 7b. The remainder of the delivery is as described above. As in the description of FIG. 4 above, spring force of the central region 735 and peripheral region 738 of gas cylinder 720 can be independently adjusted to tune the properties of the delivery pressure profile. In one preferred embodiment, the force due to central spring 735 is just sufficient to move ram 712 to the right as shown in FIG. 7b, exposing it to the pressure of peripheral region 738 of gas cylinder 720, upon which the pressure of peripheral region 738 supplies the energy for the puncture and delivery phases. This minimizes the force on ram 712 and trigger assembly 740 prior to delivery, allowing for improvements in trigger reliability and minimization of required trigger force, and minimizing creep and deformation of device components during storage.

FIG. 8 shows an embodiment conceptually very similar to that shown in FIG. 7 and described above, except that the gas pressure acts directly on the piston when ram 812 is released by trigger assembly 844 exposing gas bypass 845 and allowing the pressurized gas to flow through gas bypass 845.

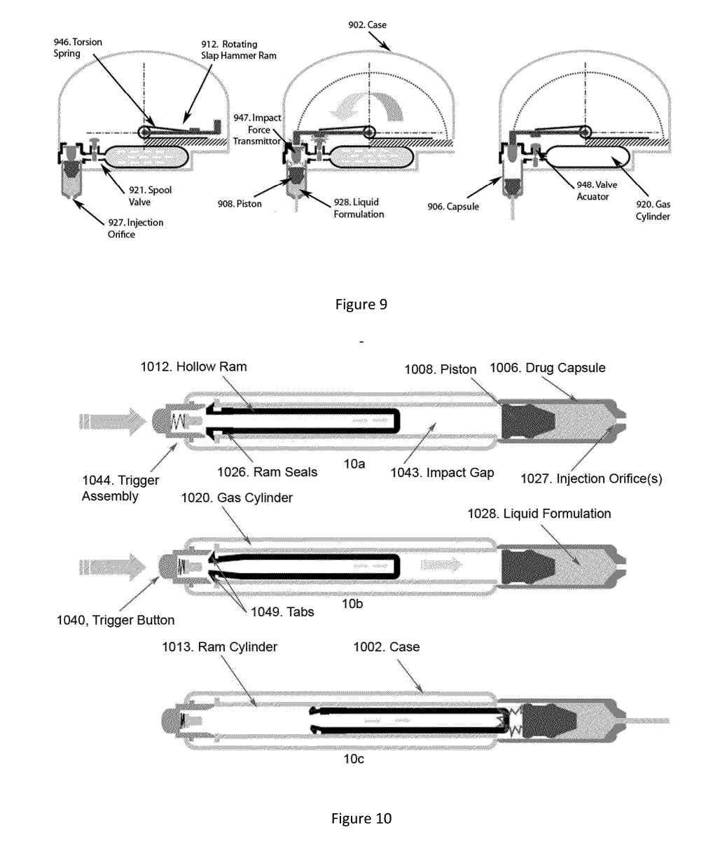

FIG. 9 shows an embodiment of the invention wherein ram 912 is a rotating slap hammer ram, with integrated timing for impact and gas release. Although this embodiment is shown with the spool valve 921 trigger embodiment, it is to be understood that it can be used with other embodiments, for example frangible gas seal valve 539. The functioning of the other components, e.g. piston 908, drug capsule 906, injection orifice 927, cap (not shown), gas cylinder 920, liquid formulation 928, and case 902 are similar to those shown in FIG. 1 as described above. FIG. 9a shows the device in the ready to fire configuration, with the orifice cap removed and any safety mechanisms in the ready to fire configuration. When the device is triggered, rotating slap hammer ram 912, urged by torsion spring 946, rotates counterclockwise as shown in FIG. 9b, striking impact force transmitting component 947, which transmits the impact force of ram 912 to piston 908, creating a pressure spike in the formulation for the puncture phase. Simultaneously, ram 912 strikes valve actuator 948, which actuates pressure valve 921, releasing the pressurized gas from gas cylinder 920, driving piston 908 downward as shown in FIG. 9c, and delivering liquid formulation 928 in the delivery phase. This embodiment has the advantages of completely decoupling the functions of impact member 912 from the delivery force, and removes any requirement for gas seals from impact member 912.

FIG. 10 shows an additional embodiment of the invention with hollow ram 1012 and rear mounted trigger assembly 1044. The functioning of the other components, e.g. piston 1008, drug capsule 1006, cap (not shown), injection orifice or orifices 1027, gas cylinder 1020, impact gap 1043, liquid formulation 1028, ram cylinder 1013 and case 1002, are similar to those shown in FIG. 1 as described above. In this embodiment, gas cylinder 1020 is an annular region outside of ram cylinder 1013, plus the interior region of hollow ram 1012. Hollow ram 1012 is urged to the right by the pressurized gas as shown in FIG. 10a, but is captured by the ram tabs 1049. FIG. 10a shows the device in the ready to fire configuration, with the orifice cap removed, and any safety mechanism (for example rotation of the trigger button 1040, see FIG. 7 and description above) in the ready to fire state. Injection orifice or orifices 1027 are pressed against the desired delivery site, and trigger button 1040 is pushed, as shown in FIG. 10b. Pressing trigger button 1040 disengages hollow ram 1012 from ram tabs 1049. In the embodiment shown in FIG. 10, this is done by deforming the end of hollow ram 1012 such that ram tabs 1049 no longer engage ram cylinder 1013, although other embodiments are possible, e.g. the end of ram cylinder 1013 is deformed outward. When hollow ram 1012 is disengaged from ram tabs 1049, it is free to travel to the right as shown in FIG. 10c, striking piston 1008 to create the pressure spike for the puncture phase, and then the pressurized gas continues to drive ram 1012 and piston 1008 through the capsule 1006, delivering formulation 1028 in the delivery phase. FIG. 10 shows ram seals 1026 on the outside of hollow ram 1012, although they can also be placed on the inside of ram cylinder 1013.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

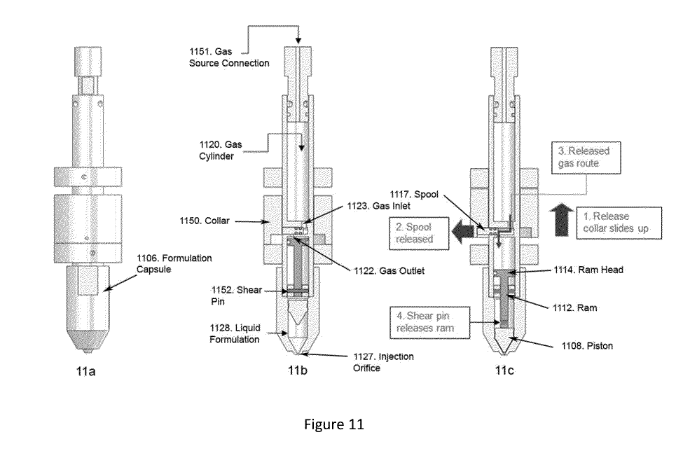

In example 1, a test was performed on a laboratory prototype with the important energizer features of the system shown in FIG. 1 and described above. An exterior view of the prototype is shown in FIG. 11a. To mimic the effect of triggering by pressing the device against the skin, simple collar 1150 is provided to release spool 1117. Formulation capsule 1106 was fabricated from steel and contained 1 mL of liquid formulation 1128, and included injection orifice 1127 with a diameter of 0.41 mm. Gas cylinder 1120 was pressurized to 60 bar via gas source connection 1151. Ram 1112 was held in place by shear pin 1152. Operation of the prototype is shown in FIG. 11c. Collar 1150 slides up, releasing spool 1117 allowing it to travel to the left as seen in 11c. This allows to the pressurized gas to flow from gas cylinder 1120, through gas inlet 1123, through the region vacated by spool 1117, and out gas outlet 1122, whereby the pressurized gas exerts a force on ram 1112 via ram head 1114. This force was sufficient to break shear pin 1152, freeing the ram to strike piston 1108 and subsequently drive liquid formulation 1128 through injection orifice 1127. The pressure profile vs. time of liquid formulation 1128 is shown in FIG. 12a. This can be compared to FIG. 12b, which shows similar data for a system of the type of system described in '086, with a formulation volume of 0.5 mL. The pressure spike of the puncture phase as shown in FIG. 12a is lower than desired (c.f. FIG. 12b). However, it can be increased by increasing the impact gap. The pressure during the delivery phase is comparable to the '086 system, and the delivery time is approximately twice as long, in line with expectations as the formulation volume is twice as large.

### Example 2

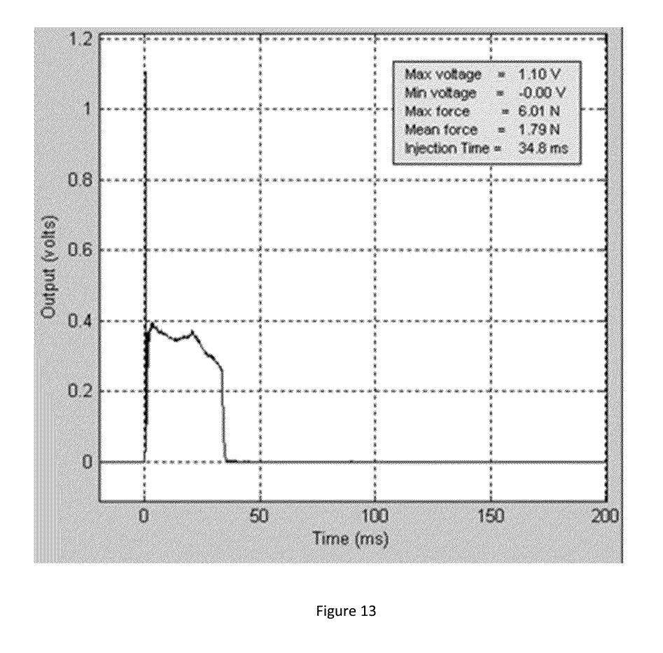

In example 2, a test was performed with a laboratory apparatus as described in example 1, but utilizing a 0.5 mL glass formulation capsule identical to that used in the '086 device. The results of this test are shown in FIG. 13, and can be seen to be quite comparable to the results from the '086 device (FIG. 12b), albeit with the same reduction in puncture phase pressure seen in example 1.

### Example 3

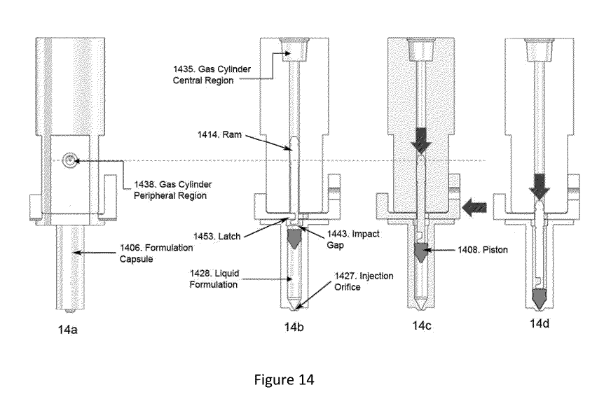

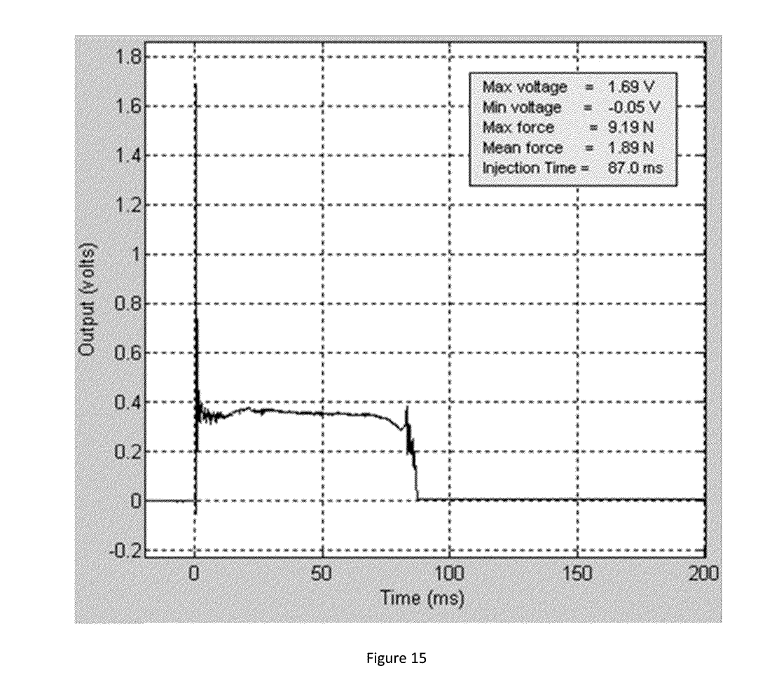

In example 3, a test was performed on a laboratory prototype (see FIG. 14a for exterior view) designed to mimic the dual gas cylinder shown in FIG. 4 and described above. The two pressures were achieved by filling gas cylinder central region 1435 with a first pressure P1, and then filling gas cylinder peripheral region 1438 with a second, lower pressure P2. Ram 1414 (note that ram seals have been omitted for clarity) was held in place using latch 1453. Formulation capsule 1406 was fabricated from steel, and contained liquid formulation 1428 in a volume of 1 mL, and included injection orifice 1427 with a diameter of 0.4 mm. Gas cylinder central region 1435 was filled to a pressure P1 of 200 MPa. Gas cylinder peripheral region 1438 was filled to a pressure P2 of 180 MPa. When latch 1453 was pushed to the right as shown in FIG. 14c, ram 1414 was released and was accelerated across impact gap 1443 under the force of the pressurized gas of gas cylinder central region 1435, and subsequently struck piston 1408 to create the pressure spike of the puncture phase. Then, as shown in FIG. 14d, ram 1414 and piston 1408 continued to be urged downward under the force of the combined pressure of the gasses of gas cylinder central region 1435 and gas cylinder peripheral region 1438, driving liquid formulation 1428 through injection orifice 1427, creating the delivery phase. FIG. 15 shows the results of a measurement of formulation pressure vs. time, and can be compared to FIG. 12b which presents the results of a similar measurement done with a device of the type described in '086, with a 0.5 mL drug formulation volume. As can be seen in FIG. 14, the system with the two pressure gas cylinder achieved a puncture phase pressure nearly identical to that with the '086 system, and thus should achieve similar sub-cutaneous injection results. The duration of the delivery phase was approximately twice as long for the dual pressure system as compared to the '086 system, as expected due to the twice as large drug formulation volume. However, the pressure during the delivery phase was nearly constant for the two pressure system, as opposed to the '086 system which showed a significant decrease in pressure during the delivery phase. Extrapolating FIG. 12b to a 1 mL delivery would suggest nearly zero pressure at the end of the delivery.

The instant invention is shown and described herein in a manner which is considered to be the most practical and preferred embodiments. It is recognized, however, that departures may be made therefrom which are within the scope of the invention and that obvious modifications will occur to one skilled in the art upon reading this disclosure.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A needle free injector, comprising:
a pressurized gas cylinder;
a spool comprising a storage seal that maintains the gas cylinder in a pressurized state during storage;
a means for releasing the spool in a manner which releases the pressurized gas into a chamber;
a ram slidably positioned in the chamber in a manner such that the ram is urged forward by released pressurized gas; and
a drug container holding a liquid drug formulation in fluid connection with a drug delivery orifice;
wherein the ram is forced to move by released pressurized gas, causing the liquid formulation to be delivered through the drug delivery orifice.

2. The needle free injector of claim 1, wherein the spool further comprises an additional seal that seals against loss of the pressurized gas after the gas has been released into the chamber.

3. The needle free injector of claim 2, wherein the spool is configured such that the pressurized gas holds the spool in a first position by a movable body which blocks motion of the spool prior to releasing the spool.

4. The needle free injector of claim 3, wherein the means for releasing the spool moves the movable body thereby exposing an end of the spool to a recess, into which recess the spool is moved by force applied by the pressurized gas.

5. The needle free injector of claim 4, wherein the movable body is moved by the act of pressing the drug delivery orifice against a surface.

6. The needle free injector of claim 5, wherein the surface is a desired injection site on human skin.

7. The needle free injector of claim 6, wherein the injector is configured such that upon releasing the spool a sub-cutaneous injection occurs forcing the liquid drug formulation out of the drug delivery orifice and through the human skin at the injection site.

8. The needle free injector of claim 7, wherein prior to releasing the spool the ram is separated by an air gap from a piston component; and
wherein the piston component is in contact with the liquid drug formulation; and
wherein the piston component seals the liquid drug in the drug container.

9. The needle free injector of claim 8, further comprising:
a cap that covers the drug delivery orifice.

10. The needle free injector of claim 9, wherein the cap must be removed prior to releasing the spool.

11. The needle free injector of claim 10, wherein the cap is removed by an act chosen from:
screw off;
break off;
click off; and
pull off.

12. The needle free injector of claim 11, wherein the cap is comprised of a first set of threads and is removed by screwing it off.

13. The needle free injector of claim 12, wherein the cap comprises an additional feature that ensures that the act of removing the cap does not accidentally trigger the device.

14. The needle free injector of claim 13, further comprising:
a case that substantially encloses the injector, the case comprising a mechanism that acts to bias the drug delivery orifice in a direction opposite that of the movement required to release the spool.

15. The needle free injector of claim 9, wherein removing the cap exposes a safety mechanism which requires actuation by an actuator prior to releasing the spool;
wherein the actuator is comprised of a lever and the lever is comprised of a tip which is captured by the cap prior to removal of the cap; and
wherein the safety mechanism comprises a blocking element that stops the movable body from moving when the drug deliver orifice is pressed against a surface, and further wherein actuating the safety mechanism via the actuator removes the blocking element.

16. (canceled)

17. (canceled)

18. The needle free injector of claim 8, wherein releasing the spool causes the pressurized gas to move the ram toward the piston and then striking the piston, causing an initial spike in liquid drug formulation pressure.

19. The needle free injector of claim 18, wherein the ram comprises a ram head characterized by a cross sectional area that is exposed to the pressurized gas when the spool is released; and
wherein the area of the ram head, the pressure of the pressurized gas, the area of the drug delivery orifice, and the length of the air gap separating the ram from the piston component are selected such that the initial spike in pressure elects liquid drug formulation out of the orifice in a manner that forms a hole into the sub-cutaneous region below the skin.

20. (canceled)

21. The needle free injector of claim 19, wherein the length of the gap is maintained during storage of the device by a holding element that holds the ram in place prior to releasing the spool and releases the ram upon releasing the spool; and
wherein the holding element releases the ram by a mechanism chosen from:
shearing;
deforming;
overcoming friction between the ram and the holding element;
being actuated by an actuator; and
a combination thereof.

22. (canceled)

23. The needle free injector of claim 8, wherein the drug container is a single component which comprises the drug delivery orifice; and
wherein the container is comprised of borosilicate glass.

24. (canceled)

25. The needle free injector of claim 5, wherein the drug delivery container comprises a nozzle component comprising the drug delivery orifice, and a separate borosilicate glass component, wherein the borosilicate glass component is a circular tube;
wherein said nozzle component is held sufficiently rigidly to the glass component that no leakage of formulation occurs during storage; and
further wherein said nozzle component is held sufficiently rigidly to the glass component that no leakage of formulation occurs when the injector is triggered.

26. The needle free injector of claim 12, wherein the cap comprises an elastomeric sealing element that seals the drug delivery orifice; and
wherein the cap further comprises:
a rotating element that eliminates strain and concomitant leakage from the elastomeric sealing element that would otherwise arise when the cap is screwed onto the device.

27. (canceled)

28. (canceled)

29. The needle free injector of claim 1, wherein the container is prefilled with 0.6 mL to about 1.6 mL of liquid drug formulation.

30. The needle free injector of claim 1, wherein the injector is prefilled with about 1.0 ml of liquid drug formulation.

31. The needle free injector of claim 14, wherein said additional feature comprises a second set of threads; and
wherein said mechanism is a set of threads that engage with said second set of threads.

32. The needle free injector of claim 31, wherein said first set of threads engage a corresponding set of threads, said corresponding set of threads are characterized by a property chosen from:
being part of the drug container;
being attached to the drug container;
being attached to a component of the needle free injector that is held rigidly at a fixed distance to the drug container.

33. (canceled)

34. A needle free injector, comprising:
a drug capsule containing a liquid drug formulation;
an orifice in the container, the orifice leading to the liquid drug formulation;
a first gas reservoir containing a first pressurized gas at a first pressure;
the first pressurized gas in contact with and urging forward a drug dispensing member;
wherein movement of the drug dispensing member is prevented by a trigger mechanism;
a second gas reservoir containing a second pressurized gas at a second pressure; and
wherein said dispensing member is not urged forward by said second pressurized gas until after it is released by said trigger mechanism.
wherein said first pressure is greater than said second pressure.
wherein said first gas reservoir is axially aligned with said dispensing member, and said second gas reservoir is displaced radially from said dispensing member.
further wherein the injector is configured such that after the drug dispensing member is released by the trigger mechanism, the drug dispensing member travels forward, exposing a gas flow path that allows the second pressurized gas to urge the drug dispensing member forward.

35-42. (canceled)

43. A needle free injector, comprising:
a drug capsule containing a liquid drug;
an orifice;
a source of energy;
a trigger mechanism comprising a ball bearing;
wherein when said trigger mechanism triggers the needle free injector, said source of energy forces the majority of said liquid drug through said at least one orifice.
wherein said source of energy comprises a spring chosen from:
a mechanical coil spring;
a Belleville washer stack; and
a compressed gas spring.

44-50. (canceled)
